(19) United States
(12) Patent Application Publication
(10) Pub. No.: US 2020/0261292 A1
(43) Pub. Date: Aug. 20, 2020

(54) PRESSURE-ELIMINATING AND PATIENT STABILIZING SUPPORT SURFACE

(71) Applicants: George Gregory STEPHENS, Trussville, AL (US); Lynn Darlene STEPHENS, Trussville, AL (US)

(72) Inventors: George Gregory STEPHENS, Trussville, AL (US); Lynn Darlene STEPHENS, Trussville, AL (US)

(21) Appl. No.: 16/430,764

(22) Filed: Jun. 4, 2019

Related U.S. Application Data
(60) Provisional application No. 62/807,161, filed on Feb. 18, 2019.

(51) Int. Cl. A61G 7/057; A61G 7/015; A61G 13/08

(52) U.S. Cl. CPC A61G 7/057; A61B 6/0407; A61G 13/08; A61G 7/015

(57) ABSTRACT

An apparatus for eliminating pressure and providing patient stability configured to be placed onto an operating room table or bed frame, or on top of another support surface on top of a bed frame. The apparatus includes a support surface that eliminates all pressure affecting the sacrum, coccyx and ischium, thereby preventing pressure ulcers affecting these anatomical sites. Additionally, the support surface substantially reduces pressures affecting the heels, thereby preventing the occurrence of pressure ulcers affecting the heels. Alternately, the support surface eliminates pressure affecting the heels, trochanter/hip and shoulder area, thereby preventing pressure ulcers affecting these anatomical sites. The support surface also provides patient stability. On an operating room table, the patient stability facilitates surgical effectiveness and safety. On a bed, the patient stability significantly reduces the need for healthcare workers to reposition or move patients up in bed, thereby significantly reducing the risks for back injuries for healthcare workers.

### CROSS-REFERENCE TO RELATED APPLICATIONS

This application claims priority from U.S. Provisional Patent Application No. 62/807,161, filed on Feb. 18, 2019 entitled “Pressure Eliminating Operating Room (OR) Table Pad,” the entire contents of which are hereby incorporated by reference.

### FIELD OF THE INVENTION

The present invention relates generally to an apparatus that can be configured for an operating room table pad or bed frame. More specifically, the present invention is a support surface that prevents pressure ulcers and stabilizes patients with the support surface being placed on top of an operating room table or a bed frame, or on top of another support surface which is placed on top of a bed frame.

### BACKGROUND

It is estimated that the annual healthcare costs directly related to pressure ulcers are about $11.6 billion. Beyond the financial implications, pressure ulcers are a source of significant morbidity and mortality for patients. Each year, about 60,000 patients die as a direct result of pressure ulcers.

It has been reported that annual direct and indirect costs due to back injuries affecting healthcare workers amounts to $20 billion. Furthermore, it has been reported that 20% of healthcare workers leave the profession due to back injuries. A major cause of back injuries for healthcare workers is moving immobile patients up in bed, i.e. moving patients toward the head end of the bed, due to the patients moving towards the foot end of the bed when the bed is raised to its normal 30 degrees bed angle. This is a potentially hazardous action that may be repeated numerous times during a healthcare worker's normal work shift.

According to the National Pressure Ulcer Advisory Panel (NPUAP), a pressure ulcer is defined as follows: a pressure ulcer is localized injury to the skin and/or underlying tissue usually over a bony prominence, as a result of pressure, or pressure in combination with shear. Thus, in accordance to definition, if there is no pressure, there is no pressure ulcer.

The sacrum, coccyx, ischium, and heels are among the bony prominences in critical locations on the human anatomy where pressure ulcers are most likely to occur when patients are placed onto operating room tables or bed with patients in the supine position. The trochanter/hip area and shoulder area are also among the bony prominences in critical locations on the human anatomy where pressure ulcers are most likely to occur when patients are placed onto operating tables in the lateral position.

The prior art, in general, has attempted to reduce the frequency of developing pressure ulcers by reducing pressure over the bony prominences (sacrum, coccyx, ischium and heels) by redistributing a portion of the force/pressure to areas of the human anatomy less vulnerable to the occurrence of pressure ulcers. Despite widespread use of the prior art support surfaces, it is estimated the mean incidence of hospital acquired pressure ulcers for U.S. Surgical/Medical ICU's is about 5.5%-6.0% for year 2017. Additionally, it has been reported about 25% of hospital acquired pressure ulcers start in the operating room.

The effectiveness of the typical prior art support surfaces with respect to the prevention of pressure ulcers is limited because these support surfaces only “reduce” pressure over the bony prominences (sacrum, coccyx, ischium and heels). Obviously, with respect to the prevention of pressure ulcers it is preferable to eliminate pressure over the bony prominences compared to the “reduction” of pressure. There is therefore the need in the art for a support surface which eliminates pressure over the bony prominences.

The typical prior art support surfaces expose healthcare workers to significant risks for back injuries since these support surfaces require the healthcare worker to frequently move patients up in bed. There is therefore a need in the art for a support surface that significantly reduces the need for healthcare workers to move patients up in bed. There is also a need in the art for a support surface that enhances patient stability while the patient is on an operating table to facilitate surgical effectiveness and safety.

### SUMMARY

According to at least one exemplary embodiment, a pressure eliminating support surface that can be configured and sized to be placed on top of an operating room table or bed frame, or on top of another support surface which is placed on top of a bed frame is disclosed. The present invention (support surface) eliminates all pressure affecting the sacrum, coccyx and ischium when the patient is laying in the supine position. Therefore, the support surface prevents pressure ulcers affecting these anatomical sites. Additionally, the support surface substantially reduces pressures affecting the heels when the patient is laying in the supine position. Therefore, the support surface substantially reduces the occurrence of pressure ulcers affecting the heels. Alternately, the support surface can be configured to eliminate all pressure affecting the heels when the patient is laying in the supine position. Therefore, the support surface can be alternately configured to prevent pressure ulcers affecting the heels. Moreover, the support surface eliminates all pressure affecting the trochanter/hip area when the patient is laying in the lateral position. Therefore, the support surface prevents pressure ulcers affecting these anatomical sites. The support surface can also be alternately configured to eliminate all pressure affecting the shoulder area when the patient is laying in the lateral position. Therefore, the support surface can also prevent pressure ulcers affecting the shoulder area. Further, the support surface enhances patient stability while the patient is on an operating table or in a bed. For an operating room table, the enhanced patient stability provided by the support surface facilitates surgical effectiveness and safety. For a bed, the enhanced patient stability provided by the support surface significantly reduces the need for healthcare workers to reposition or move patients up in bed, or towards the head end of the bed, thereby significantly reducing the risks for back injuries for healthcare workers.

### BRIEF DESCRIPTION OF THE DRAWINGS

Advantages of embodiments of the present invention will be apparent from the following detailed description of the exemplary embodiments. The following detailed description should be considered in conjunction with the accompanying figures in which:

FIG. 1 illustrates an exemplary embodiment of the present invention concerning the operating table configuration for the pressure eliminating and patient stabilizing support surface.

FIG. 2 illustrates two alternate embodiments of the operating table configuration for the pressure eliminating and patient stabilizing support surface, one of which eliminates all pressure affecting the heels and the other eliminates all pressure affecting the shoulders.

FIG. 3 illustrates an exemplary embodiment of the present invention concerning the bed frame configuration for the pressure eliminating and patient stabilizing support surface.

FIG. 4 illustrates an alternate embodiment of the bed frame configuration for the pressure eliminating and patient stabilizing support surface, which eliminates all pressure affecting the heels.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description, discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments”, or “invention” do not require that all embodiments of the invention include the discussed feature, advantage, or mode of operation.

As used herein, the word “patient” designates a person who is positioned on the support surface. However, the intention is not to limit the use of the support surface a person under medical supervision, as the support surface may be used by any individual.

The present invention is an apparatus that prevents pressure ulcers on patients positioned on operating tables or beds for substantial periods of time. Specifically, the present invention is a support surface that is placed on top of an operating table or a bed frame, or on top of another support surface which is placed on top of a bed frame. The present invention works by eliminating all pressure affecting the sacrum, coccyx, ischium and the heels. These bony prominences are the critical locations on the human anatomy where pressure ulcers are most likely to occur when patients are positioned for an extended time on an operating room table or on a bed in the supine position. The present invention also works by eliminating all pressure affecting the trochanter/hip area and shoulder area. These bony prominences are the critical locations on the human anatomy where pressure ulcers are most likely to occur when patients are positioned for an extended time on an operating room table in the lateral position.

The support surface is constructed of multi layers whereby the layers consist of visco-elastic foam and base foam. The top layer or layers of the support surface are constructed of visco-elastic foam with the bottom layer constructed of a firmer base foam. The majority of the support surface thickness is constructed of a soft to moderately soft visco-elastic foam, which may have, for example, an indentation load deflection (ILD) ranging from 11 to 20 ILD, such as 11 to 13 ILD or 16 to 18 ILD. This provides an important collapsibility feature which is discussed in detail below. Alternate relative visco-elastic foam thickness and ILD's or alternate materials of construction can be used as long as they provide the important collapsibility feature. The firmer base foam may have an indentation load deflection ranging from 40 to 50 ILD, such as 44 ILD.

The support surface includes an aperture within the multi-layers. The aperture is located in a position that corresponds to the location of an individual's buttocks when the individual is placed on top of the support surface in the supine position. When an individual is laying on the support surface in the supine position, the individual's sacrum, coccyx and ischium are comfortably suspended over air within the aperture, thereby eliminating pressure affecting these bony prominences and thereby preventing pressure ulcers from occurring on these anatomical sites. Alternately, the support surface may include apertures within the multi layers that corresponds to the location of an individual's heels when the individual is placed on top of the support surface in the supine position. As a result, the individual's heels are comfortably suspended in air thereby eliminating pressure affecting the heels and thereby preventing pressure ulcers from occurring on the heels. Also, alternately, the support surface may include an aperture within the multi layers that corresponds to the location of an individual's shoulder area when the individual is placed on top of the support surface in the lateral position. As a result, the individual's shoulder area is comfortably suspended in air thereby eliminating pressure affecting the shoulder area and thereby preventing pressure ulcers from occurring on the shoulder area. The previously mentioned aperture in the location of the individual's buttocks also comfortably suspends the individual's trochanter/hip area over air within the aperture when the individual is laying on the support surface in the lateral position thereby eliminating pressure affecting these bony prominences and thereby preventing pressure ulcers from occurring on the trochanter/hip area. The multi-layers (visco-elastic foam and base foam) of the support surface allow the support surface to be collapsible in the vicinity of the aperture to the extent that the sacrum, coccyx and ischium are suspended in air over the aperture but allowing the patient's buttocks to extend significantly down into the aperture when the individual is laying on the support surface in the supine position.

This collapsibility feature at the support surface aperture located at the patient's buttocks area provides at least four important benefits. First, the collapsibility feature provides enhanced stability for the patient by helping to prevent the patient from moving sideways across the support surface and also helping to prevent the patient from moving down towards the foot end of the support surface when the patient is placed onto the support surface in the supine position. This enhanced stability significantly reduces the need for healthcare workers to reposition or move patients up in bed. Therefore, the support surface significantly reduces the risks for back injuries for healthcare workers. Furthermore, the enhanced stability is especially important to facilitate surgical effectiveness and safety. Second, the collapsibility feature provides a comfortable suspension of the patient's sacrum, coccyx and ischium over air within the aperture, and thereby preventing the sacrum, coccyx and ischium from contacting any surface when the patient is placed onto the support surface in the supine position. Third, with the patient in the supine position, the collapsibility feature allows the patient's buttocks to significantly extend down into the aperture which causes the patient's heels to elevate, or off load, which substantially reduces pressures affecting the heels, and thereby substantially reducing the occurrence of pressure ulcers affecting the heels. Fourth, the enhanced stability provided by the collapsibility feature also reduces shear and friction forces on the patient's skin outside the aperture, thus further reducing the risk of developing pressure ulcers in areas of the skin outside the aperture. The collapsibility feature also is applicable with respect to the alternate apertures regarding enhanced stability and also regarding comfortable suspension of the relevant bony prominences over air within the apertures and thereby preventing pressure ulcers from affecting the relevant anatomical sites.

The material from which the support surface is made also provides additional benefits. The top layer or layers of the support surface are constructed of visco-elastic foam. The substantial use of a soft to moderately soft visco-elastic foam, representing the majority of the support surface thickness, provides at least two important benefits. First, the visco-elastic foam has fluid like properties which enable the edges of the support surface aperture to roll inward toward the aperture when a patient is laying on the support surface. This “rolling in” increases support surface area in contact with the patient's skin, thereby significantly decreasing pressures at the edges of the aperture. Second, with the patient in the supine position, the combination of the visco-elastic foam construction and the support surface aperture further reduces pressures experienced by the patient in areas of the buttocks outside the aperture. This reduced pressure in the buttocks area outside the support surface aperture is due to increased support surface immersion and envelopment in this area induced by increased forces experienced in this area due to the support surface aperture. Increased support surface immersion and envelopment decrease pressure in the buttocks area outside the aperture by increasing the area of the support surface in contact with the patient's skin.

In some exemplary embodiments, the support surface includes an optional support surface plug that can alternately be inserted into the support surface aperture. The support surface plug materials of construction are exactly the same as the support surface, consisting of multi-layers of visco-elastic foam and base foam. The support surface plug dimensions are exactly the same as the aperture dimensions.

The support surface plug provides at least three benefits for patients in the supine position. First, the support surface plug provides unique versatility whereby mobile patient's that have little, or no, risk pressure ulcer risk could use the support surface with the plug inserted. Immobile patients that have significant pressure ulcer risk, or existing pressure ulcers, would use the support surface without the plug inserted and with the aperture open. Second, with the plug inserted, the patient would experience a high level of comfort due to the visco-elastic foam construction. Third, also with the plug inserted into the aperture there would be a significant increase in support surface immersion and envelopment at the patient's sacrum, coccyx and ischium areas, thereby significantly decreasing pressures affecting these areas. In some embodiments, the support surface may further have a cover, which is compatible for hospital or nursing home use, to avoid direct contact between the patient and the support surface.

In some embodiments, the support surface is sized to fit over a conventionally-sized operation table or within a conventionally-sized bed frame, such as those normally found in a hospital or nursing home. Moreover, in these embodiments, the support surface is sized to accept typical covers or sheets used for operation tables or hospital or nursing home beds.

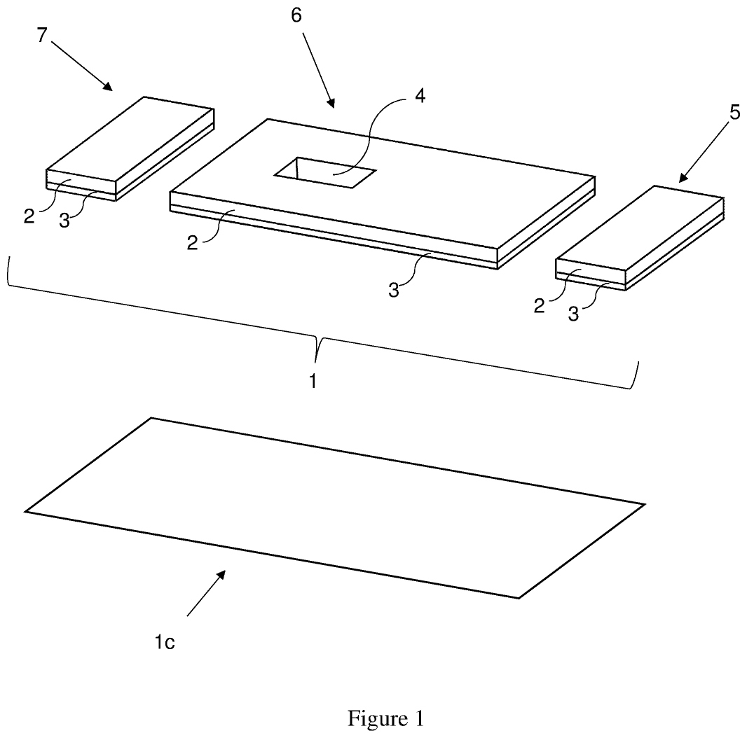

In some exemplary embodiments, the support surface is configured and sized for an operating room table, as shown in FIG. 1.

The operating table configuration 1 may include three sections: a head section 5, a middle section 6 and a foot section 7. Dimensions for the various sections may include, for example: a head section 5 of approximately 9 inches to 10 inches in length by approximately 19 inches to 21 inches in width by approximately 4 inches to 5 inches in thickness, a middle section 6 of approximately 40 inches to 42 inches in length by approximately 19 inches to 21 inches in width by approximately 4 inches to 5 inches in thickness, and a foot section 7 of approximately 19 inches to 21 inches in length by approximately 19 inches to 21 inches in width by approximately 4 inches to 5 inches in thickness. In at least one exemplary embodiment, the dimension may include a head section 5 of approximately 9 inches in length by approximately 20 inches in width by approximately 5 inches in thickness, a middle section 6 of approximately 40 inches in length by approximately 20 inches in width by approximately 5 inches in thickness, and a foot section 7 of approximately 20 inches in length by approximately 20 inches in width by approximately 5 inches in thickness.

Each of the head section 5, the middle section 6 and the foot section 7 is made from at least a top layer of the visco-elastic foam 2, representing the majority of the support surface thickness, and a layer base foam 3.

The support surface major aperture 4 for the operating room table configuration 1 is located in the middle section 6 at a position which corresponds to where the patient's buttocks is to be placed so that the patient's sacrum, coccyx and ischium are suspended over air within the major aperture 4 when the patient is laying on the support surface in the supine position. For example, the position of the major aperture 4 is located at the center of the middle section 6 with respect to the width dimension of middle section 6 and with the end of the major aperture 4 located approximately 3 inches to 6 inches from the end of middle section 6 at the end of middle section 6 where the patient's legs would be located. Additionally, this major aperture 4 corresponds to where the patient's trochanter/hip area is to be placed so that these anatomical sites will also be suspended over air within the major aperture 4 when the patient is laying on the support surface in the lateral position. The support surface major aperture 4 dimensions for the operating room table configuration 1 described above are as follows: the length is approximately 10 inches to 11 inches, the width is approximately 5 inches to 6 inches, and the depth is approximately 4 inches to 5 inches. The length dimension of the aperture is in line with the length dimension of the middle section 6 or measured along the length of the operating room table configuration 1. In at least one exemplary embodiment, the major aperture 4 has a length of approximately 11 inches, a width of approximately 6 inches, and a depth of 5 inches.

The operating room table configuration 1 may optionally have a cover 1c compatible with operating room table use.

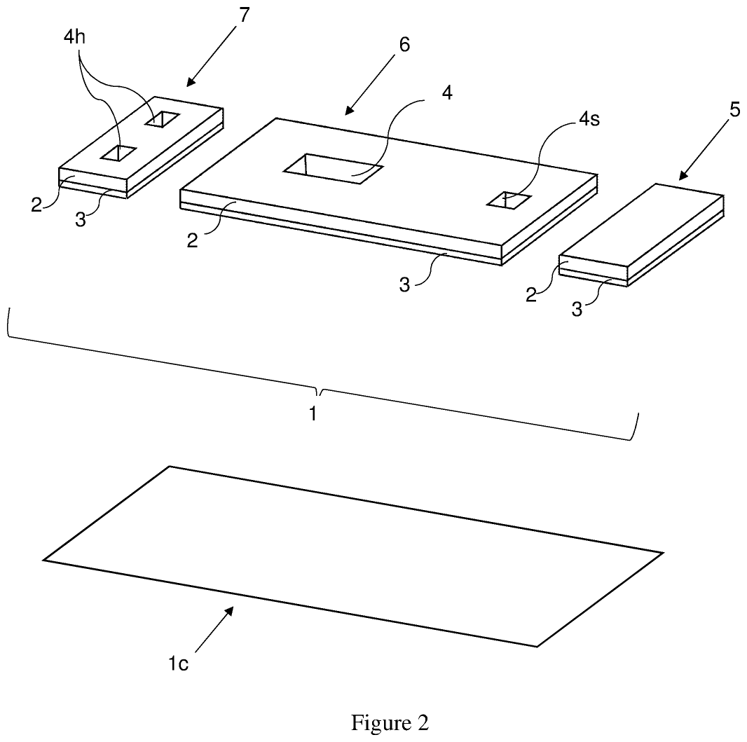

Alternately, as illustrated in FIG. 2, the middle section 6 of the operating room table configuration 1 may include a minor aperture 4s corresponding to the location of the patient's shoulder area so that the patient's shoulder area will be comfortably suspended in air within the minor aperture 4s when the patient is laying on the support surface in the lateral position. Although FIG. 2 shows both major aperture 4 and minor aperture 4s in the middle section 6, in at least one embodiment the middle section 6 shown in FIG. 2 may only include minor aperture 4s. The dimensions for this minor aperture 4s are approximately as follows: 4 inches to 5 inches length, width and depth. In at least one exemplary embodiment the dimensions for the minor aperture 4s are approximately 5 inches in length, width and depth.

Alternately, as also illustrated in FIG. 2, the foot section 7 of the operating room configuration 1 may include a pair of twin apertures 4h that correspond to the locations of the patient's heels so that the patient's heels will be comfortably suspended in air within the pair of twin apertures 4h when the patient is laying on the support surface in the supine position. Although FIG. 2 shows the pair of twin apertures 4h in the foot section 7 in combination with major aperture 4 and minor aperture 4s in the middle section 6, the foot section 7 may have the pair of twin apertures 4h without the middle section 6 having either the major aperture 4 or minor aperture 4s. The dimensions of each of the twin apertures 4h are approximately as follows: 5 inches to 7 inches length×3 inches to 4 inches width×4 inches to 5 inches depth. The length dimension is in line with the length dimension for the foot section. In at least one exemplary embodiment, the dimensions of each of the twin apertures 4h are 5 inches length×3 inches width×5 inches depth.

In other exemplary embodiments, the operating room table configuration 1 shown in FIGS. 1 and 2, may include only one section, the middle section 6, or the middle section 6 and head section 5, or middle section 6 and foot section 7, or middle section 6, head section 5 and foot section 7. The dimensions for the various sections, the major aperture 4, the minor aperture 4s, the pair of twin apertures 4h, and the multi-layer of foams are the same as described above and illustrated in FIG. 1 and FIG. 2.

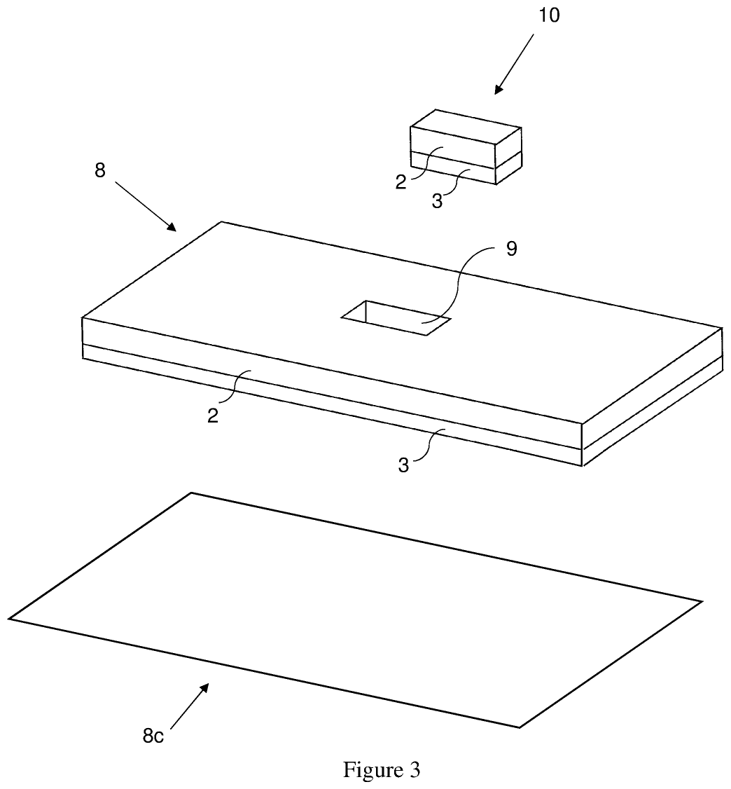

In some exemplary embodiments, the support surface is configured and sized for a bed frame, as illustrated in FIG. 3. The bed frame configuration 8 for the support surface has the following dimensions: the length is approximately 76 inches to 80 inches, the width is approximately 33 inches to 36 inches, and the thickness is approximately 6 to 10 inches. It at least one exemplary embodiment, the dimensions of the bed frame configuration 8 are has the following dimensions: the length is approximately 80 inches, the width is approximately 36 inches, and the thickness is approximately 8 inches. The support surface central aperture 9 for the bed frame configuration has the following dimensions: the length is approximately 10 inches to 11 inches, the width is approximately 5 inches to 6 inches, and the depth is approximately 6 to 10 inches. The length dimension of the central aperture 9 is in line with the length dimension of the bed frame configuration 8. It at least one exemplary embodiment, the central aperture 9 has a length of approximately 11 inches, a width of approximately 6 inches, and a depth of approximately 8 inches The central aperture 9 for the bed frame configuration 8 for the support surface is located near the center of the support surface. Similar to the operating table configure 1, bed frame configuration 8 also includes the visco-elastic foam 2, representing the majority of the support surface thickness, and a layer base foam 3.

The bed frame configuration 8 may optionally have a cover 8c compatible with typical hospital or nursing home use.

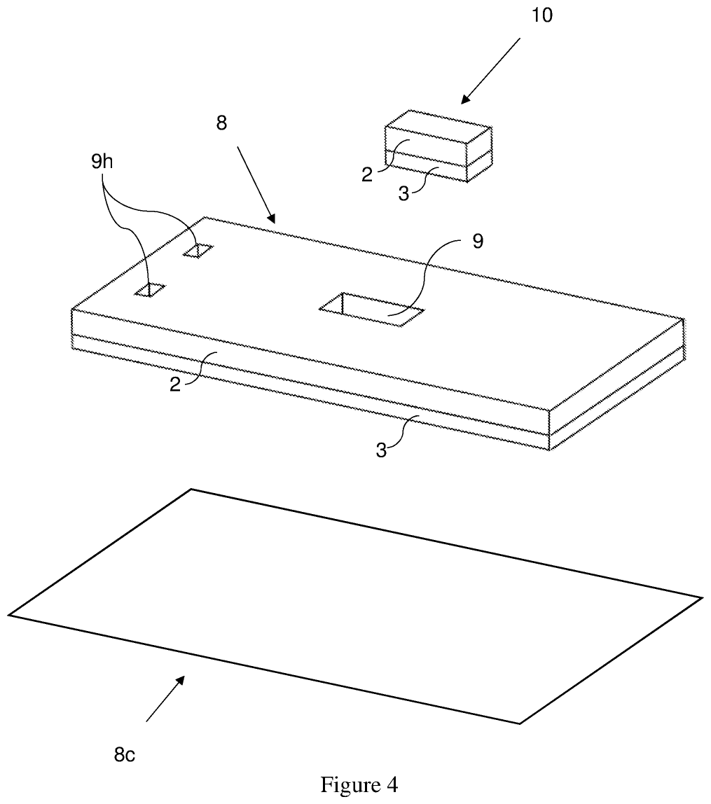

Alternately, as illustrated in FIG. 4, the bed frame configuration 8 may include a pair of end apertures 9h located at the foot end of the support surface at the patient's heels, in additional to the other features described in FIG. 3. Although FIG. 4 shows the pair of end apertures 9h in combination with the central aperture 9, in at least one embodiment the bed frame configuration 8 may have the pair of end apertures 9h without the central aperture 9. The dimensions of each of the end apertures 9h is as follows: approximately 5 inches to 7 inches length×approximately 3 inches to 4 inches width×approximately 6 to 10 inches depth. The length dimension is in line with the length dimension for the support surface for the bed frame configuration 8. In at least one exemplary embodiment, the dimensions of each of the end apertures 9h are: approximately 5 inches length×approximately 3 inches width×approximately 8 inches depth.

The bed frame configuration 8 for the support surface may include an optional support surface plug 10, as shown in both FIGS. 3 and 4, that can alternately be inserted into the support surface central aperture 9 located at the buttocks area. The optional support surface plug 10 shares the same structure as the rest of the support surface, i.e. it also includes the visco-elastic foam 2, representing the majority of the support surface plug 10 thickness, and the layer base foam 3. The optional support surface plug 10 could be used for individuals that are mobile with little, or no, pressure ulcer risk. Additionally, these mobile individuals would have the ability to move themselves up in bed, if necessary, and/or significantly help the caregiver in moving the individual up in bed.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. An apparatus for eliminating pressure and providing patient stability on an operating room table, comprising:
a support surface comprising:
at least a top layer of visco-elastic foam and a base layer of foam, the base layer foam being a firmer foam than the visco-elastic foam, and
a major aperture extending through the support surface, the aperture having a location that corresponds to (i) where a patient's sacrum, coccyx and ischium are to be located when a patient lays over the support surface in the supine position and (ii) where a patient's trochanter/hip are to be located when a patient lays over the support surface in the lateral position,
wherein the combination of the at least one top layer visco-elastic foam and base layer of firmer foam of the support surface allow the support surface to be collapsible proximate to the major aperture,
the support surface being collapsible proximate to the major aperture enables:
(i) a patient's sacrum, coccyx and ischium to be suspended in air over the major aperture to allow a patient's buttock to extend downward into the major aperture and cause a patient's heels to elevate, thereby eliminating pressure affecting the sacrum, coccyx and ischium, reducing pressure affecting the heels for a patient laying on the support surface, providing enhanced stability of a patient laying on the support surface by preventing movement, and reducing shear and friction forces on a patient's skin outside the major aperture, and
(ii) a patient's trochanter/hip to be suspended in air over the major aperture to allow a patient's trochanter/hip to extend downward into the major aperture, thereby eliminating pressure affecting the trochanter/hip, providing enhanced stability of a patient laying on the support surface by preventing movement, and reducing shear and friction forces on a patient's skin outside the major aperture.

2. The apparatus of claim 1, wherein the support surface has a cover compatible with operating room table use.

3. The apparatus of claim 1, wherein the support surface has a length of approximately 40 inches to 42 inches, a width of approximately 19 inches to 21 inches, and a thickness of approximately 4 inches to 5 inches.

4. The apparatus of claim 1, wherein the major aperture has a length of approximately 10 inches to 11 inches, a width of approximately 5 inches to 6 inches, and a depth of approximately 4 inches to 5 inches, and the length of the aperture is measured along a length of the support surface.

5. An apparatus for eliminating pressure and providing patient stability on an operating room table, comprising:
a support surface comprising:
at least a top layer of visco-elastic foam and a base layer of foam, the base layer foam being a firmer foam than the visco-elastic foam, and
a minor aperture extending through the support surface, the minor aperture having a location that corresponds to where a patient's shoulder is to be located when a patient lays over the support surface in the lateral position,
wherein the combination of the at least one top layer visco-elastic foam and base layer of firmer foam of the support surface allow the support surface to be collapsible proximate to the minor aperture,
the support surface being collapsible proximate to the minor aperture enables a patient's shoulder to be suspended in air over the minor aperture to allow a patient's shoulder to extend downward into the minor aperture, thereby eliminating pressure affecting the shoulder, providing enhanced stability of a patient laying on the support surface by preventing movement, and reducing shear and friction forces on a patient's skin outside the minor aperture.

6. The apparatus of claim 5, wherein the support surface has a cover compatible with operating room table use.

7. The apparatus of claim 5, wherein the support surface has a length of approximately 40 inches to 42 inches, a width of approximately 19 inches to 21 inches, and a thickness of approximately 4 inches to 5 inches.

8. The apparatus of claim 5, wherein the minor aperture has a length of approximately 4 inches to 5 inches, a width of approximately 4 inches to 5 inches, and a depth of approximately 4 inches to 5 inches.

9. An apparatus for eliminating pressure and providing patient stability on an operating room table, comprising:
a support surface comprising:
at least a top layer of visco-elastic foam and a base layer of foam, the base layer foam being a firmer foam than the visco-elastic foam, and
two twin apertures extending through the support surface, the twin apertures having locations that correspond to where a patient's heels are to be located when a patient lays over the support surface in the supine position,
wherein the combination of the at least one top layer visco-elastic foam and base layer of firmer foam of the support surface allow the support surface to be collapsible proximate to the twin apertures,
the support surface being collapsible proximate to the twin apertures enables a patient's heels to be suspended in air over the twin apertures to allow a patient's heels to extend downward into the twin apertures, thereby eliminating pressure affecting the heels.

10. The apparatus of claim 9, wherein the support surface has a cover compatible with operating room table use.

11. The apparatus of claim 10, wherein the support surface has a length of approximately 19 inches to 21 inches, a width of approximately 19 inches to 21 inches, and a thickness of approximately 4 inches to 5 inches.

12. The apparatus of claim 9, wherein each of the twin apertures has a length of approximately 5 inches to 7 inches, a width of approximately 3 inches to 4 inches, and a depth of approximately 4 inches to 5 inches.

13. An apparatus for eliminating pressure and providing patient stability configured to be placed onto a bed frame, or on top of another support surface on top of a bed frame, comprising:
a support surface comprising:
at least a top layer of visco-elastic foam and a base layer of foam, the base layer foam being a firmer foam than the visco-elastic foam, and
a central aperture extending through the support surface and proximate to a center location of the support surface and corresponds to where a patient's sacrum, coccyx and ischium are to be located when a patient lays over the support surface,
wherein the combination of the at least one top layer visco-elastic foam and base layer of firmer foam of the support surface allow the support surface to be collapsible proximate to the central aperture,
the support surface being collapsible proximate to the central aperture enables a patient's sacrum, coccyx and ischium to be suspended in air over the central aperture to allow a patient's buttock to extend downward into the central aperture and cause a patient's heels to elevate, thereby eliminating pressure affecting the sacrum, coccyx and ischium, reducing pressure affecting the heels for a patient laying on the support surface, providing enhanced stability of a patient laying on the support surface by preventing movement, and reducing shear and friction forces on a patient's skin outside the central aperture.

14. The apparatus of claim 13, wherein the support surface further comprises a removably insertable support surface plug for the support surface central aperture, and the support surface plug is formed of the at least a top layer of visco-elastic foam and the base layer of foam of the support surface, dimensions of the support surface plug are identical to dimensions of the support surface central aperture, and the support surface provides support surface immersion and envelopment at a location corresponding to a patient's sacrum, coccyx and ischium areas, thereby significantly decreasing pressures affecting the areas.

15. The apparatus of claim 13, wherein the support surface further comprises a cover compatible with typical hospital or nursing home use.

16. The apparatus of claim 13, wherein the support surface a has a length of approximately 76 inches to 80 inches, a width of approximately 33 inches to 36 inches, and a thickness of approximately 6 to 10 inches.

17. The apparatus of claim 13, wherein the central aperture has a length of approximately 10 inches to 11 inches, a width of approximately 5 inches to 6 inches, and a depth thickness of approximately 6 to 10 inches, and the length of the central aperture is along a length of the support surface.

18. An apparatus for eliminating pressure and providing patient stability configured to be placed onto a bed frame, or on top of another support surface on top of a bed frame, comprising:
a support surface comprising:
at least a top layer of visco-elastic foam and a base layer of foam, the base layer foam being a firmer foam than the visco-elastic foam, and
two end apertures extending through the support surface, the end apertures having locations that correspond to where a patient's heels are to be located when a patient lays over the support surface in the supine position,
wherein the combination of the at least one top layer visco-elastic foam and base layer of firmer foam of the support surface allow the support surface to be collapsible proximate to the two end apertures, the support surface being collapsible proximate to the two end apertures enables a patient's heels to be suspended in air over the two end apertures to allow a patient's heels to extend downward into the two end apertures, thereby eliminating pressure affecting the heels.

19. The apparatus of claim 18, wherein the support surface further comprises a cover compatible with typical hospital or nursing home use.

20. The apparatus of claim 18, wherein the support surface a has a length of approximately 76 inches to 80 inches, a width of approximately 33 inches to 36 inches, and a thickness of approximately 6 to 10 inches.

21. The apparatus of claim 18, wherein each of the end apertures has a length of approximately 5 inches to 7 inches, a width of approximately 3 inches to 4 inches, and a depth of approximately 6 to 10 inches, and the length of each of the end apertures is along a length of the support surface.
